(19) United States
(12) Patent Application Publication
(10) Pub. No.: US 2019/0254637 A1
(43) Pub. Date: Aug. 22, 2019

(54) ULTRASONIC OBSERVATION DEVICE, AND METHOD FOR OPERATING ULTRASONIC OBSERVATION DEVICE

(71) Applicant: OLYMPUS CORPORATION, Tokyo (JP)

(72) Inventor: Tomohiro SABATA, Tokyo (JP)

(73) Assignee: OLYMPUS CORPORATION, Tokyo (JP)

(21) Appl. No.: 16/402,339

(22) Filed: May 3, 2019

Related U.S. Application Data
(63) Continuation of application No. PCT/JP2017/038993, filed on Oct. 27, 2017.

(30) Foreign Application Priority Data
Nov. 9, 2016 (JP) 2016-219161

(51) Int. Cl. A61B 8/08; A61B 8/14; A61B 8/06

(52) U.S. Cl. CPC A61B 8/5223; A61B 8/481; A61B 8/08; A61B 8/06; A61B 8/5276; A61B 8/14

(57) ABSTRACT

An ultrasonic observation device includes a processor including hardware. The processor is configured to: read a video made of ultrasonic images arranged in a chronological manner from a memory; perform time intensity curve (TIC) analysis on the video based on temporal changes in signal intensities of ultrasonic waves reflected by a contrast dye administered into a subject being examined; extract information about user operation stored in the memory along the ultrasonic images; and control a display to display fact that the information about user operation is extracted.

This application is a continuation of PCT International Application No. PCT/JP2017/038993 filed on Oct. 27, 2017, which designates the United States, incorporated herein by reference, and which claims the benefit of priority from Japanese Patent Application No. 2016-219161, filed on Nov. 9, 2016, incorporated herein by reference.

### BACKGROUND

The present disclosure relates to an ultrasonic observation device, and a method for operating an ultrasonic observation device.

Typically, an ultrasonic observation device is known that transmits ultrasonic waves to the subject being examined; receives the ultrasonic waves that get reflected from the inside of the subject being examined; and accordingly generates ultrasonic images. Moreover, a technology (for example, refer to Japanese Laid-open Patent Publication No. 2012-210507) is known in which, using the ultrasonic observation device, the signal intensities of the ultrasonic waves that get reflected by a contrast dye administered into the subject being examined are stored in the form of a video made of chronologically-arranged ultrasonic images, and time intensity curve (TIC) analysis is performed with respect to the stored video; and the blood flow inside the subject being examined is detected from the inflow speed and the outflow speed of the contrast dye inside the subject being examined. Then, the user, such as a doctor, becomes able to make use of the information about the blood flow inside the subject being examined in diagnosing the subject being examined.

### SUMMARY

An ultrasonic observation device according to one aspect of the present disclosure includes a processor including hardware, the processor being configured to: read a video made of ultrasonic images arranged in a chronological manner from a memory; perform time intensity curve (TIC) analysis on the video based on temporal changes in signal intensities of ultrasonic waves reflected by a contrast dye administered into a subject being examined; extract information about user operation stored in the memory along the ultrasonic images; and control a display to display fact that the information about user operation is extracted.

The above and other features, advantages and technical and industrial significance of this disclosure will be better understood by reading the following detailed description of exemplary embodiments of the disclosure, when considered in connection with the accompanying drawings.

### BRIEF DESCRIPTION OF THE DRAWINGS

FIG. 1 is a block diagram illustrating a configuration of an ultrasonic observation system, which includes an ultrasonic observation device, according to an exemplary embodiment;

FIG. 2 is a flowchart for explaining a brief overview of the operations performed in the ultrasonic observation device for storing a video according to an exemplary embodiment;

FIG. 3 is a flowchart for explaining a brief overview of the operations performed in the ultrasonic observation device for performing time intensity curve (TIC) analysis according to an exemplary embodiment;

FIG. 4 is a diagram illustrating an exemplary image displayed in a display device;

FIG. 5 is a diagram illustrating a situation in which an indicator is superimposed on a TIC graph area;

FIG. 6 is a diagram illustrating an exemplary image displayed in the display device;

FIG. 7 is a flowchart for explaining a brief overview of the operations performed in the ultrasonic observation device for storing a video according to an exemplary embodiment;

FIG. 8 is a flowchart for explaining a brief overview of the operation performed in the ultrasonic observation device for performing the TIC analysis according to an exemplary embodiment;

FIG. 9 is a diagram illustrating a situation in which operation timings of user operations are superimposed on the TIC graph area;

FIG. 10 is a diagram illustrating an exemplary image displayed in the display device;

FIG. 11 is a diagram illustrating a situation in which the TIC analysis is performed for the interval between the operation timings at which user operations were performed; and

FIG. 12 is a diagram illustrating an exemplary image displayed in the display device.

### DETAILED DESCRIPTION

Exemplary embodiments of an ultrasonic observation device, an ultrasonic observation system, a method for operating an ultrasonic observation device, and a computer-readable recording medium according to the present disclosure are described below with reference to the accompanying drawings. However, the present disclosure is not limited by the embodiments described below. The present disclosure can be implemented generally in ultrasonic observation devices that are capable of performing the TIC analysis.

Meanwhile, in the drawings, identical or corresponding elements are referred to by the same reference numerals. Moreover, each drawing is schematic in nature, and it needs to be kept in mind that the relationships among the dimensions of the elements or the ratio of the elements may be different than the actual situation. Among the drawings too, there may be portions having different relationships among the dimensions or having different ratios.

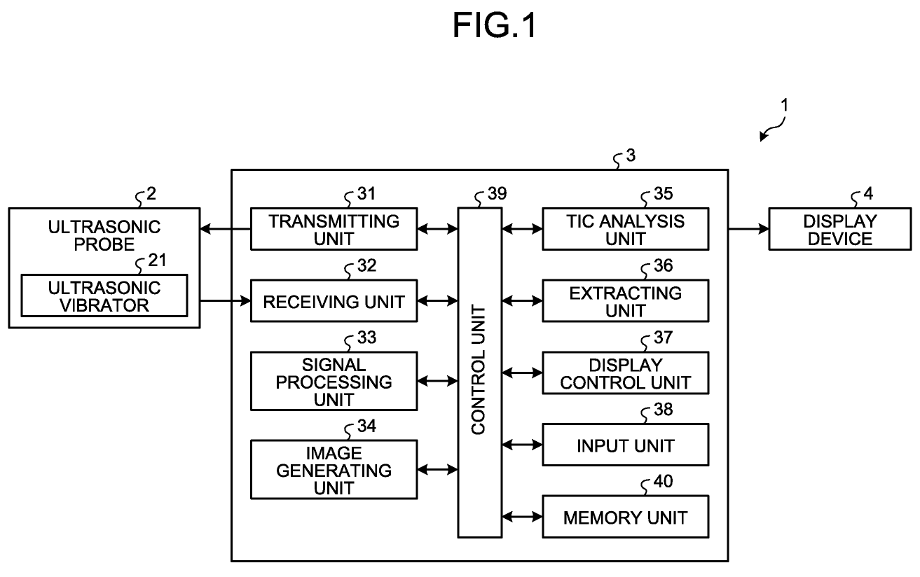

FIG. 1 is a block diagram illustrating a configuration of an ultrasonic observation system, which includes an ultrasonic observation device, according to an exemplary embodiment. An ultrasonic observation system 1 includes an ultrasonic probe 2 that transmits ultrasonic waves to the subject being examined who represents the observation target and receives the ultrasonic waves that get reflected from the subject being examined; includes an ultrasonic observation device 3 that generates ultrasonic images based on the ultrasonic signals obtained by the ultrasonic probe 2; and a display device 4 that displays the ultrasonic images displayed by the ultrasonic observation device 3. In the ultrasonic observation device 3, the following two modes are made selectable as observations modes for observing the ultrasonic waves: a B mode meant for generating images by converting the amplitude of echo signals into luminance; and a contrast radiography mode meant for generating images in which the contrast agent for ultrasonic waves, which represents a suspension of microbubbles introduced into the observation target, is displayed in a highlighted manner.

The ultrasonic probe 2 is an endoscope that includes, at the front end thereof, an ultrasonic vibrator 21 which converts electrical pulse signals received from the ultrasonic observation device 3 into ultrasonic pulses (acoustic pulses) and irradiates the subject being examined representing the observation target with the ultrasonic pulses; and which converts the ultrasonic echo reflected from the subject being examined into electrical echo signals (ultrasonic signals) expressed using voltage change, and outputs the electrical echo signals. The ultrasonic vibrator 21 is implemented using a convex array vibrator that transmits and receives ultrasonic waves in a radial fashion along a plurality of sound ray directions. The ultrasonic probe 2 either can be configured to mechanically scan the ultrasonic vibrator 21, or can be configured to have an array of a plurality of elements installed as the ultrasonic vibrator 21 and can electrically scan the ultrasonic vibrator 21 by electronically switching the elements to be used in transmission and reception and by delaying the transmission and reception of each element.

The ultrasonic probe 2 usually includes an imaging optical system and an imaging element; is inserted into the digestive canal (the esophagus, the stomach, the duodenum, and the large intestine) or into the respiratory apparatus (the trachea and the bronchial tube) of the subject being examined; and is capable of taking images of the digestive canal, the respiratory apparatus, and the nearby organs (the pancreas, the gallbladder, the bile duct, the biliary tract, the lymph node, the mediastinal space, and the blood vessels). Moreover, the ultrasonic probe 2 includes a light guide that guides the illumination light with which the subject being examined is irradiated at the time of taking images. Of the light guide, the front end reaches the front end of the inserted portion of the ultrasonic probe 2 inside the subject being examined, and the proximal end is connected to a light source device that generates the illumination light.

The ultrasonic observation device 3 includes a transmitting unit 31, a receiving unit 32, a signal processing unit 33, an image generating unit 34, a TIC analysis unit 35, an extracting unit 36, a display control unit 37, an input unit 38, a control unit 39, and a memory unit 40.

The transmitting unit 31 is electrically connected to the ultrasonic probe 2; and transmits transmission signals (pulse signals), which are made of high-voltage pulses, to the ultrasonic vibrator 21 based on predetermined waveforms and transmission timings. Herein, the frequency band of the pulse signals that are transmitted by the transmitting unit 31 is preferably set to a wide band that nearly covers the linear response frequency band of the electroacoustic conversion of the pulse signals into ultrasonic pulses in the ultrasonic vibrator 21. Moreover, the transmitting unit 31 transmits various control signals, which are output by the control unit 39, to the ultrasonic probe 2.

The receiving unit 32 receives echo signals that represent electrical reception signals from the ultrasonic vibrator 21; performs analog-to-digital (A/D) conversion of the echo signals and generates data of digital radio frequency (RF) signals (hereinafter, called RF data); and outputs the RF data. Moreover, the receiving unit 32 also has the function of receiving a variety of information containing an identifier (ID) from the ultrasonic probe 2 and sending the information to the control unit 39.

The signal processing unit 33 generates digital B-mode reception data based on the RF data received from the receiving unit 32. More particularly, the signal processing unit 33 performs known operations such as bandpass filter, envelope demodulation, and logarithmic transformation with respect to the RF data; and generates the digital B-mode reception data. In the logarithmic transformation, common logarithm equal to the amount obtained by dividing the RF data with a reference voltage Vc is taken and is expressed as a digital value. The signal processing unit 33 outputs the B-mode reception data corresponding to one frame to the image generating unit 34. Herein, the signal processing unit 33 is implemented using a central processing unit (CPU) or using various arithmetic circuits.

The image generating unit 34 generates image data based on the RF data received from the receiving unit 32. The image generating unit 34 performs signal processing such as scan converter processing, gain processing, and contrast processing with respect to the B-mode reception data stored in the memory unit 40, as well as performs thinning of data according to the data step width decided based on the image display range in the display device 4; and generates ultrasonic images representing B-mode image data. In the scan converter processing, the scanning direction for the B-mode reception data is converted from the scanning direction for ultrasonic waves into the display direction of the display device 4. Herein, a B-mode image is a grayscale image in which the values of R (red), G (green), and B (blue), which represent the variables when the RGB color coordinate system is adapted as the color space, are matched to each other.

The image generating unit 34 performs coordinate transformation for performing resequencing so as to enable correct spatial expression of the scanning range in the B-mode reception data received from the signal processing unit 33 and then performs interpolation among the sets of B-mode reception data so as to fill the gaps among them, and generates B-mode image data.

The TIC analysis unit 35 reads the video made of the chronologically-arranged ultrasonic images that are stored in the memory unit 40, and performs the TIC analysis based on the temporal changes in the signal intensities of the ultrasonic waves that get reflected by the contrast dye administered into the subject being examined.

The extracting unit 36 extracts information about user operations that is stored along with the ultrasonic images in the memory unit 40. The information about user operations represents operation information that contains the timings, the types, and the details of the operations performed by the user. Moreover, the user operations include an operation of changing the gain or the contrast that affects the ultrasonic images; an operation of changing the display range for ultrasonic images that affects the display in the display device 4 and scrolling the ultrasonic images; and an operation of setting a measurement cursor in the ultrasonic images and writing a comment.

The display control unit 37 outputs, to the display device 4, the ultrasonic images generated by the image generating unit, the result of the TIC analysis performed by the TIC analysis unit 35, and image signals displaying a variety of information; and controls the display in the display device 4. Moreover, the display control unit 37 displays, in the display device 4, the fact that the extracting unit 36 extracted user operations.

The input unit 38 receives signals based on input operations performed using a user interface such as a keyboard, buttons, a mouse, a trackball, a touch-sensitive panel, or a touch-pad. The input unit 38 receives input of a user instruction for starting the contrast radiography mode or ending the contrast radiography mode. Moreover, the input unit 38 receives input of a user instruction for changing the transmission condition regarding the transmission of ultrasonic waves from the ultrasonic vibrator 21. Examples of the transmission condition include the strength of the output of the ultrasonic waves from the ultrasonic vibrator 21.

The control unit 39 controls the ultrasonic observation system 1 in entirety. The control unit 39 is implemented using a CPU or various arithmetic circuits having a calculation function and a control function. The control unit 39 reads the information stored in the memory unit 40, and comprehensively controls the ultrasonic observation device 3 by performing various arithmetic operations related to the method for operating the ultrasonic observation device 3. Meanwhile, the control unit 39 can also be configured using the same CPU that is commonly used for the signal processing unit 33, the image generating unit 34, the TIC analysis unit 35, the extracting unit 36, and the display control unit 37.

The memory unit 40 is used to store various computer programs that are meant for operating the ultrasonic observation system 1, and to store data containing various parameters required in the operations of the ultrasonic observation system 1. Moreover, the memory unit 40 is used to store videos made of chronologically-arranged ultrasonic images and to store the information about user operations that is stored along with the ultrasonic images.

The memory unit 40 is used to store various computer programs including an operation program that is meant for implementing a method for operating the ultrasonic observation system 1. The operation program can be stored, for extensive distribution, in a computer-readable recording medium such as a hard disk, a flash memory, a compact disk read only memory (CD-ROM), a digital versatile disk read only memory (DVD-ROM), or a flexible disk. Alternatively, the abovementioned various computer programs can be downloaded via a communication network. The communication network is implemented using, for example, an existing public line, a local area network (LAN), or a wide area network (WAN); and can be configured in either a wired manner or a wireless manner.

The memory unit 40 having such a configuration is implemented using a read only memory (ROM) in which various programs are installed in advance and using a random access memory (RAM) in which operation parameters and data of various operations are stored.

The display device 4 receives data signals of the ultrasonic images, which are generated by the ultrasonic observation device 3, via a picture cable, and displays the data signals. The display device 4 is configured using a monitor such as a liquid crystal display or an organic electro luminescence (organic EL) display.

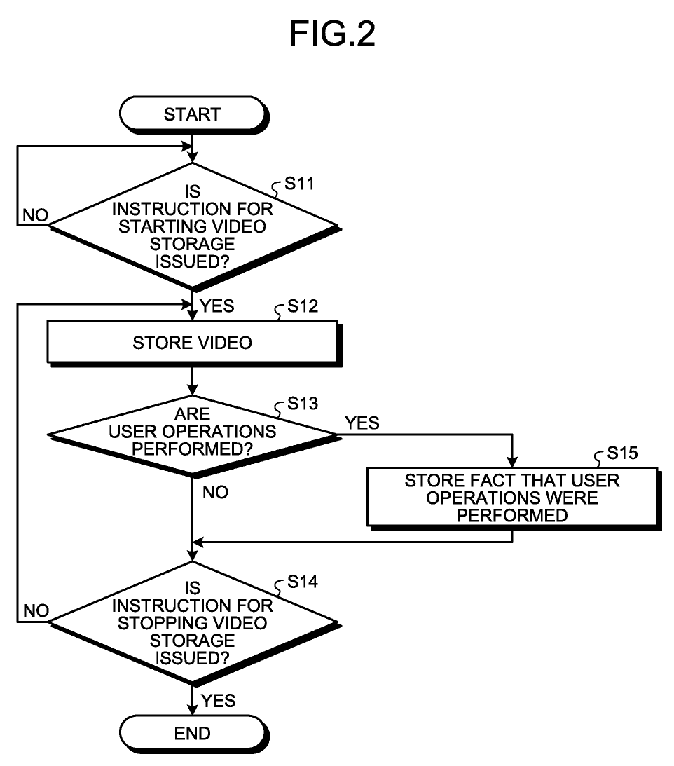

Given below is the explanation of the operations performed in the ultrasonic observation device 3 for storing a video. FIG. 2 is a flowchart for explaining a brief overview of the operations performed in the ultrasonic observation device for storing a video according to this embodiment. Firstly, the control unit 39 of the ultrasonic observation device 3 determines whether or not an instruction for starting the video storage has been issued (Step S11). If no instruction is issued yet (No at Step S11), the control unit 39 performs the operation at Step S11 in a repeated manner.

When an instruction is issued (Yes at Step S11), the control unit 39 starts storing the video in the memory unit 40 (Step S12).

If no user operation is performed during the video storage (No at Step S13), the control unit 39 determines whether or not an instruction for stopping the video storage is issued (Step S14). If no instruction is issued yet (No at Step S14), the system control returns to Step S12 and the video storage in the memory unit 40 is carried on. When an instruction is issued (Yes at Step S14), the control unit 39 ends the video storage in the memory unit 40. That marks the end of the operations.

Meanwhile, if user operations are performed during the video storage (Yes at Step S13), the control unit 39 stores, in the memory unit 40, the fact that user operations were performed (Step S15). More particularly, a flag indicating that user operations were performed is generated and is stored in a predetermined memory area. Then, at Step S14, the determination for the end of the video storage is performed, and accordingly the operation is either ended or carried on.

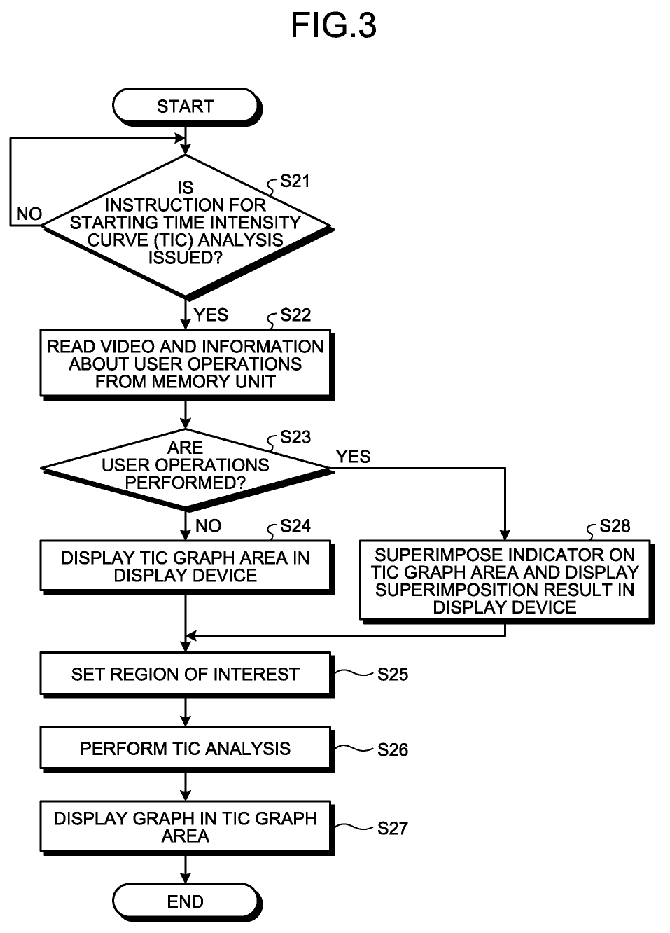

Given below is the explanation of the operations performed in the ultrasonic observation device 3 for performing the TIC analysis. FIG. 3 is a flowchart for explaining a brief overview of the operations performed in the ultrasonic observation device for performing the TIC analysis according to this embodiment. Firstly, the control unit 39 of the ultrasonic observation device 3 determines whether or not an instruction for starting the TIC analysis is issued (Step S21). If no instruction is issued yet (No at Step S21), the control unit 39 performs the operation at Step S21 in a repeated manner.

When an instruction is issued (Yes at Step S21), the control unit 39 reads a video and the information about user operations from the memory unit 40 (Step S22). The information about user operations contains information about the flag that is generated in the memory unit 40 to indicate that user operations were performed.

Then, the control unit 39 determines whether or not the information read from the memory unit 40 contains the fact that user operations were performed (Step S23). More particularly, the control unit 39 determines whether or not the extracting unit 36 has extracted a flag indicating that user operations were performed. If it is determined that user operations were not performed (No at Step S23), the control unit 39 displays, in the display device 4, a TIC graph area in which the result of the analysis performed by the TIC analysis unit 35 is displayed as a graph (Step S24). Subsequently, when the user sets a region of interest (ROI) in the ultrasonic image (Step S25) and issues a predetermined instruction for starting the TIC analysis, then the TIC analysis unit 35 performs the TIC analysis (Step S26). Then, the display control unit 37 displays the result of the TIC analysis as a graph in the TIC graph area in the display device 4 (Step S27).

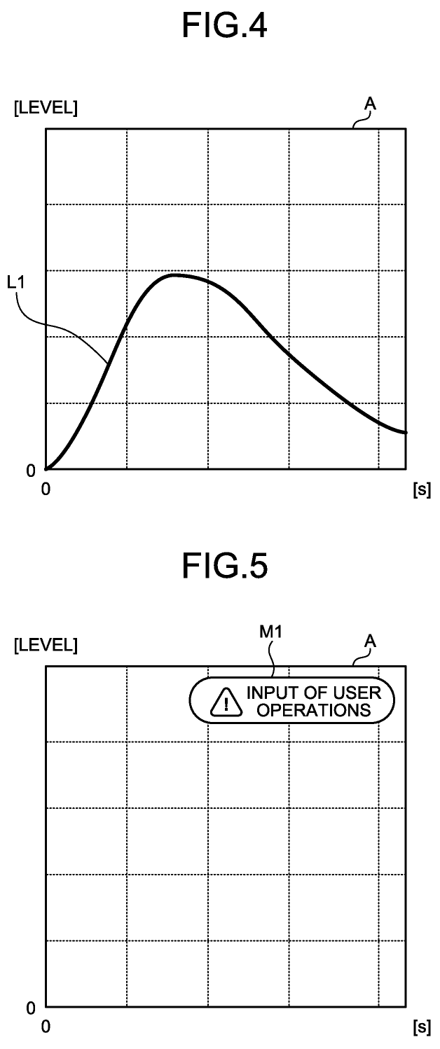

FIG. 4 is a diagram illustrating an exemplary image displayed in the display device. When no user operation is performed; for example, a graph displaying the result of the TIC analysis as illustrated in FIG. 4 is displayed in a TIC graph area A in the display device 4. When no user operation is performed, the result of the TIC analysis can be correctly obtained as illustrated by a line L1 in FIG. 4.

Meanwhile, if it is determined that user operations were performed (Yes at Step S23), then the control unit 39 superimposes, on the TIC graph area A, an indicator for indicating that user operations were performed and displays the result in the display device 4 (Step S28). FIG. 5 is a diagram illustrating a situation in which an indicator is superimposed on the TIC graph area. As illustrated in FIG. 5, in the TIC graph area A, a warning display M1 is displayed as an indicator for indicating that user operations were performed. As a result, the user becomes able to know that user operations were performed; and, as soon as the TIC graph A is displayed, it becomes possible to determine not to perform the TIC analysis.

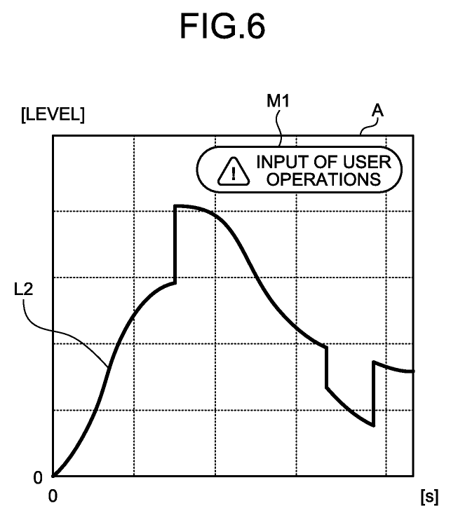

Meanwhile, if the user still continues with the TIC analysis, the operations from Step S25 to Step S27 are performed, and a graph is displayed in the TIC graph area A. FIG. 6 is a diagram illustrating an exemplary image displayed in the display device. When user operations are performed; for example, a graph displaying the result of the TIC analysis as illustrated in FIG. 6 is displayed in the display device 4. As illustrated in a line L2 in FIG. 6, when user operations are performed, the TIC analysis cannot be correctly performed and there are times when the line L2 undergoes changes in a discontinuous manner at the operation timings at which user operations are performed. In this case too, as illustrated in FIG. 6, the warning display M1 is displayed in the graph, and the user becomes able to know that user operations were performed. As a result, the user can take measures such as again performing the TIC analysis in the sections in which user operations were not performed.

The ultrasonic observation device 3 according to another exemplary embodiment has an identical configuration to the configuration illustrated in FIG. 1, but the operations performed therein are different. Hence, the same explanation is not given again.

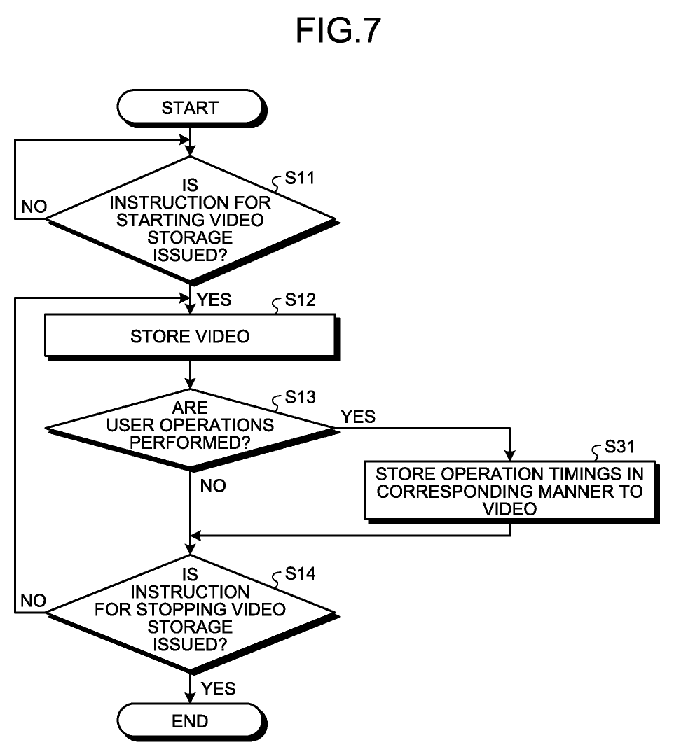

FIG. 7 is a flowchart for explaining a brief overview of the operations performed in the ultrasonic observation device for storing a video according to this embodiment. As illustrated in FIG. 7, firstly, the ultrasonic observation device 3 performs the operations from Step S11 to Step S14 in an identical manner to the above embodiment.

If user operations are performed during the video storage (Yes at Step S13), then the control unit 39 stores the operation timings, at which the user operations were performed in the video, in a corresponding manner to the video (Step S31). More particularly, flags indicating the user timings, at which user operations were performed, are generated and are stored in a predetermined memory area in a corresponding manner to the respective ultrasonic images included in the video.

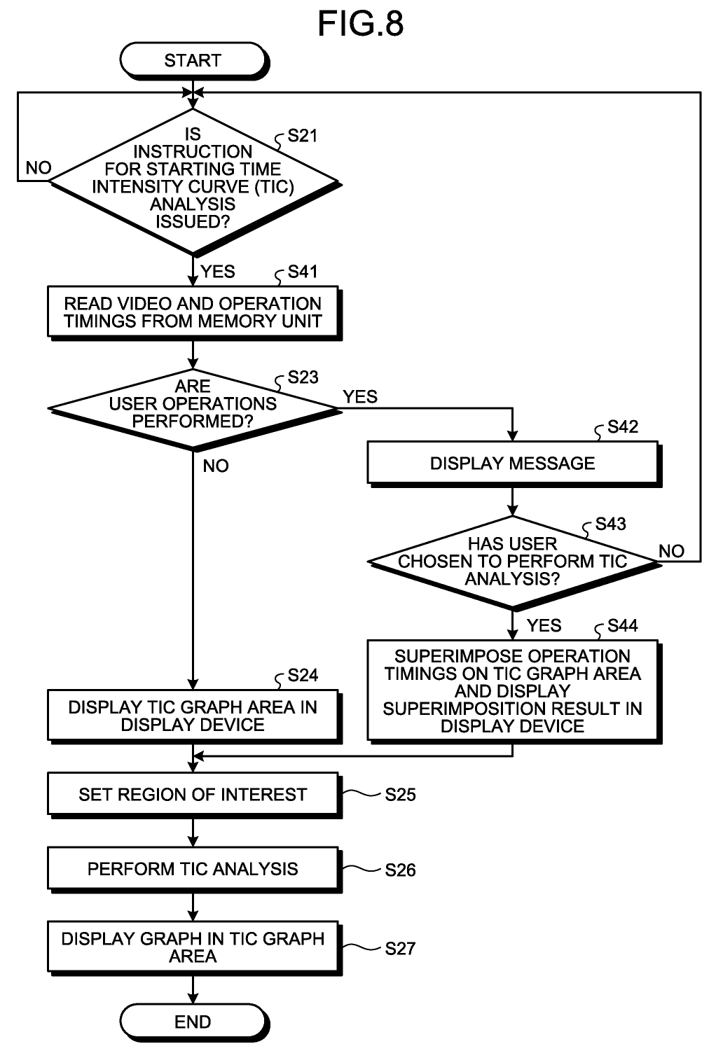

FIG. 8 is a flowchart for explaining a brief overview of the operation performed in the ultrasonic observation device for performing the TIC analysis according to this embodiment. As illustrated in FIG. 8, firstly, the ultrasonic observation device 3 performs the operations from Step S21 to Step S27 in an identical manner to the above embodiment. At Step S41, the control unit 39 reads the video as well as the operation timings from the memory unit 40. More particularly, the control unit 39 reads, from the memory unit 40, the ultrasonic images included in the video along with a graph indicating the operation timings corresponding to the ultrasonic images.

If the control unit 39 determines that user operations were not performed (No at Step S23), then the operations from Step S24 to Step S27 are performed, so that the result of the TIC analysis as illustrated in FIG. 4 can be obtained.

When it is determined that user operations were performed (Yes at Step S23), the control unit 39 instructs the display control unit 37 to display, in the display device 4, a message for checking with the user about whether or not to perform the TIC analysis even though user operations were performed (Step S42). More particularly, the display control unit 37 displays a message such as “Would you like to perform the TIC analysis even though user operations were performed?” in the display device 4.

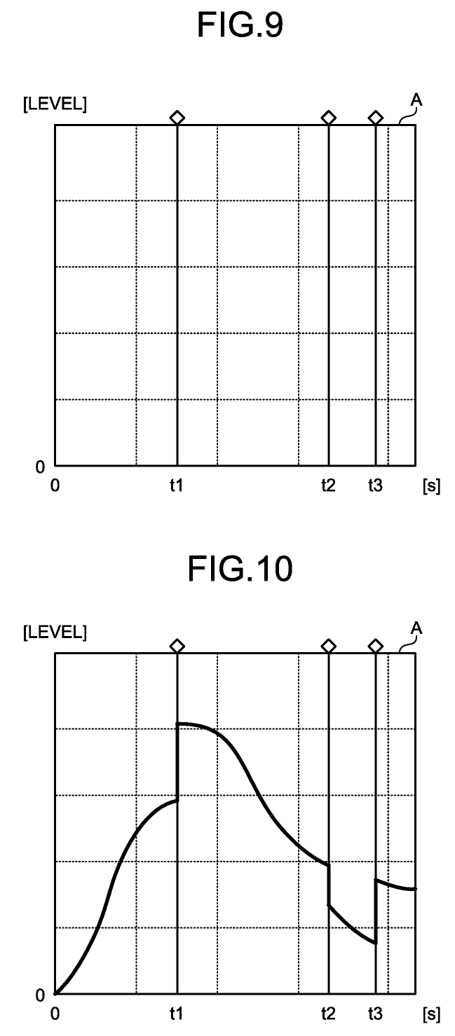

If the user choses to perform the TIC analysis (Yes at Step S43), then the control unit 39 superimposes the operation timings of the user operations on the TIC graph area A and then displays the TIC graph area A in the display device 4 (Step S44). FIG. 9 is a diagram illustrating a situation in which the operation timings of user operations are superimposed on the TIC graph area. As illustrated in FIG. 9, the display control unit 37 displays an image in which operation timings tl to t3 are superimposed using, for example, straight lines in the TIC graph area A. As a result, the user becomes able to know that user operations were performed; and, as soon as the TIC graph A is displayed, it becomes possible to determine not to perform the TIC analysis.

Meanwhile, if the user choses to continue with the TIC analysis, then the operations from Step S25 to Step S27 are performed and a graph is displayed in the TIC graph area A. FIG. 10 is a diagram illustrating an exemplary image displayed in the display device. As illustrated in FIG. 10, the operation timings tl to t3 are displayed in the graph, and thus the user becomes able to know that user operations were performed.

Meanwhile, at Step S43, if the user chooses not to perform the TIC analysis, then the system control returns to Step S21 and the subsequent operations are performed.

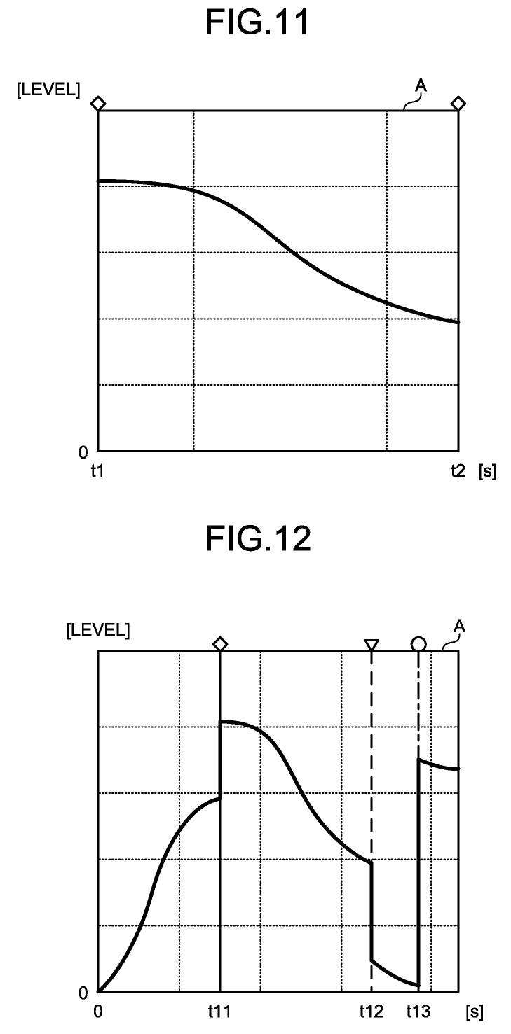

Meanwhile, the TIC analysis unit 35 can be configured to have a function by which the interval between neighboring operation timings across the selected point of time in the TIC graph area A is automatically set as the TIC analysis range. FIG. 11 is a diagram illustrating a situation in which the TIC analysis is performed for the interval between the operation timings at which user operations were performed. As illustrated in FIG. 11, when the user selects the interval between the operation timings t1 and t2 with reference to FIG. 9 and sets the region of interest; the TIC analysis unit 35 performs the TIC analysis for that interval, and the control unit 39 instructs the display control unit 37 to generate an image in which the result of the TIC analysis in the concerned interval is illustrated as a graph. Meanwhile, the configuration can be such that, even when the user selects the interval between the operation timings t1 and t2 after the TIC analysis is performed for all intervals is illustrated in FIG. 10, the operations are performed in an identical manner.

Moreover, the TIC analysis unit 35 can be configured to have a function by which the interval from the start of storing till the first user operation is automatically set as the TIC analysis range. Furthermore, the TIC analysis unit 35 can be configured to have a function by which, according to the selection made by the user, the interval from the start of storing till the first user operation is set as the TIC analysis range.

### MODIFICATION EXAMPLE

FIG. 12 is a diagram illustrating an exemplary image displayed in the display device. As illustrated in FIG. 12, according to the types of the operations, the display control unit 37 notifies the operation timings t11 to t12 of user operations by distinguishing them according to, for example, line types, line colors, and symbols. As a result, the user not only becomes able to know the user timings of user operations but also becomes able to know the types of the operations.

Moreover, the display control unit 37 can be configured to have a function for notifying the ultrasonic images and the types of user operations. Because of this function, the user becomes able to confirm the types of user operations performed when the ultrasonic image being viewed was taken. More particularly, in an ultrasonic image, a plurality of icons is displayed side-by-side corresponding to the user operations such as gain variation and contrast variation. The icons of the user operations that were performed at the time when the concerned ultrasonic image was taken are displayed in a brighter (whiter) tone, and the other icons are displayed in a darker (blacker) tone. Moreover, in an ultrasonic image, numerical values (parameters) indicating the gain and the contrast are displayed. Thus, the numerical values corresponding to the user operations that were performed when the concerned ultrasonic image was taken can be displayed in a brighter (whiter) tone and the other numerical values can be displayed in a darker (blacker) tone.

Meanwhile, although the explanation herein is given about an ultrasonic endoscope as an example, the ultrasonic observation device according to the present disclosure can be implemented in an external ultrasonic probe that performs ultrasonic irradiation from the surface of the body of the subject being examined. An external ultrasonic probe is usually used for the observation of the abdominal organs (the liver, the gallbladder, and the urinary bladder), or the breasts (particularly, the mammary gland), or the thyroid gland.

Meanwhile, the ultrasonic observation system 1 can be configured to also have the functions as described below.

The ultrasonic observation system 1 can be configured to enable observation in an elastography mode. In the elastography mode, information related to the hardness of the observation target in the set area (hereinafter, also called an elastography region of interest (ROI)) is obtained; and color information corresponding to the hardness is superimposed onto an ultrasonic image representing a B-mode image (to obtain an elastography image). Moreover, the ultrasonic observation system 1 can be configured to display, in the display device 4, a histogram of relative strain values in the region of interest (i.e., a strain histogram).

Furthermore, the ultrasonic observation system 1 can be configured to calculate statistical values (the average value and the dispersion value) from the strain histogram.

Moreover, the ultrasonic observation system 1 can be configured to store the strain histogram as computerized system validation (CSV).

Furthermore, at the time of storing the strain histogram as computerized system validation, the ultrasonic observation system 1 can be configured to store the strain histogram in a corresponding manner to the ultrasonic image for which the region of interest was set.

Moreover, the ultrasonic observation system 1 can be configured to enable arbitrary setting (presetting) of the initial size of the region of interest at the time of measuring the strain histogram.

Furthermore, at the time of measuring the strain histogram in a repeated manner for a plurality of number of times, the ultrasonic observation system 1 can be configured to have the function for keeping the same shape and the same size as in the previous region of interest.

Moreover, when a touch-pad is connected as the input device, the ultrasonic observation system 1 can be configured to enable making changes in the size of the region of interest according to pinch-in/pinch-out operations.

Furthermore, when various statistical values are calculable from the strain histogram, the ultrasonic observation system 1 can be configured to enable selection of the statistical values to be displayed in the display device 4.

Moreover, the ultrasonic observation system 1 can be configured to enable displaying, in real time in the display device 4, the central depth and the size of the region of interest during the measurement of the strain histogram.

Furthermore, the ultrasonic observation system 1 can be configured to enable setting of two regions of interest and to enable measurement of the strain ratio representing the ratio of strain values in the two regions of interest. Moreover, the ultrasonic observation system 1 can be configured to store the measurement result as computerized system validation.

Furthermore, at the time of storing the measurement result of the strain ratio as computerized system validation, the ultrasonic observation system 1 can be configured to store the measurement result in a corresponding manner to the ultrasonic image for which the regions of interest were set.

Moreover, the ultrasonic observation system 1 can be configured to enable arbitrary setting (presetting) of the initial size of each of the two regions of interest at the time of measuring the strain ratio.

Furthermore, at the time of measuring the strain ratio in a repeated manner for a plurality of number of times, the ultrasonic observation system 1 can be configured to have the function for keeping the same shape and the same size as in the previous regions of interest.

Moreover, when a touch-pad is connected as the input device, the ultrasonic observation system 1 can be configured to enable making changes in the size of the regions of interest according to pinch-in/pinch-out operations.

Furthermore, when various statistical values are calculable from the strain ratio, the ultrasonic observation system 1 can be configured to enable selection of the statistical values to be displayed in the display device 4.

Moreover, the ultrasonic observation system 1 can be configured to enable displaying, in real time in the display device 4, the central depth and the size of the regions of interest during the measurement of the strain ratio.

Furthermore, at the time of superimposing an elastography image on an ultrasonic image, the ultrasonic observation system 1 can be configured to enable making changes in the ratio by which either the ultrasonic image or the elastography image becomes easier to view.

Moreover, the ultrasonic observation system 1 can be configured to enable making changes in the coloration of the elastography image. More particularly, the ultrasonic observation system 1 can be configured to enable making changes in the coloration from, for example, the coloration in which the blue color represents hardness and the red color represents softness to the coloration in which the red color represents hardness and the blue color represents softness.

Furthermore, during the observation in the elastography mode, when a freezing operation is performed, the ultrasonic observation system 1 can be configured to have a function for displaying automatically-colored images (strain-less images or images excluding the images deleted according to error determination).

Moreover, when a freezing operation is performed, the ultrasonic observation system 1 can be configured to display, in a simultaneous manner in the display device 4, a strain graph indicating the time course of strain values, the image colored at the time of freezing, and a plurality of colored images of the past. At that time, the ultrasonic observation system 1 can be configured to display, in the display device 4, the points of time on the time axis of the strain graph to which the colored images of the past correspond. Furthermore, when a desired position in the strain graph is selected, the ultrasonic observation system 1 can be configured to have a function for displaying the colored image corresponding to the selected position.

Moreover, the ultrasonic observation system 1 can be configured to have a function by which, when a freezing operation is performed, an image group belonging to a particular section (enclosed in a frame) is displayed in a highlighted manner from a list of colored images.

Furthermore, the ultrasonic observation system 1 can be configured to have a function by which, when a freezing operation is performed, the colored image at the time of freezing and the colored image selected from a plurality of colored images of the past are displayed in a simultaneous manner in the display device 4.

Moreover, the ultrasonic observation system 1 can be configured to have a function by which, when the strain graph, the colored image at the time of freezing, and the colored images of the past are being displayed in the display device 4, if a single colored image is selected (by a double click, for example), the selected colored image is displayed across the entire display device 4.

Furthermore, the ultrasonic observation system 1 can be configured to have a function by which, when a freezing operation is performed, a list of only the colored images is displayed in the display device 4.

Moreover, the ultrasonic observation system 1 can be configured to have a function by which, when a freezing operation is performed, the display can be switched between the display of a list of only the colored images and the single-screen display of the colored images.

Furthermore, the ultrasonic observation system 1 can be configured to have a function by which, when a freezing operation is performed, only the colored images can be reproduced.

Moreover, the ultrasonic observation system 1 can be configured to have a function for adjusting the persistence of elastography images.

Furthermore, the ultrasonic observation system 1 can be configured to have a function for adjusting the noise-cut level of elastography images.

Moreover, the ultrasonic observation system 1 can be configured to have a function for adjusting the smoothness in the scan line direction of the elastography images (i.e., a smoothing function).

Furthermore, the ultrasonic observation system 1 can be configured to have a function that enables setting of the initial size and the initial display position of the elastography region of interest.

Moreover, the ultrasonic observation system 1 can be configured to enable making changes in the size and the position of the elastography region of interest during the reproduction of the stored images.

Furthermore, during the reproduction of the stored images, when an elastography image is to be superimposed onto an ultrasonic image, the ultrasonic observation system 1 can be configured to enable making changes in the ratio by which either the ultrasonic image or the elastography image becomes easier to view.

Moreover, during the reproduction of the stored images, the ultrasonic observation system 1 can be configured to enable calculation of the strain histogram.

Furthermore, during the reproduction of the stored images, the ultrasonic observation system 1 can be configured to enable calculation of the strain ratio.

Moreover, in the contrast radiography mode, the ultrasonic observation system 1 can be configured to have a function by which transmission-reception of ultrasonic pulses for contrast radiography and display of images in the display device 4 is performed in an intermittent manner.

Moreover, the ultrasonic observation system 1 can be configured to have a THE mode in which the pulse output is adjusted to cater to the contrast radiography mode.

Furthermore, the ultrasonic observation system 1 can be configured to have a function for superimposing contrast radiography images onto a fundamental image and then displaying the superimposition result in the display device 4.

Moreover, at the time of superimposing contrast radiography images onto a fundamental image, the ultrasonic observation system 1 can be configured to have a threshold value set for the luminance of contrast radiography images, so that only the contrast radiography images having the luminance equal to or greater than the threshold value are superimposed onto the fundamental image and the superimposition result is displayed.

Furthermore, the ultrasonic observation system 1 can be configured to have a function by which, when a timer is turned ON, video recording is automatically started in tandem.

Moreover, the ultrasonic observation system 1 can be configured to have a function for storing the timing from a timer in a corresponding manner to video storage in the memory unit 40.

Furthermore, the ultrasonic observation system 1 can be configured to have a function by which, when a freezing operation is performed, the timing of freezing and the timing of the timer continuing with the counting are displayed in the display device 4.

Moreover, in order to hold down the depletion of the contrast dye, the ultrasonic observation system 1 can be configured to have a function for putting a restriction to ensure that, even if the frame rate is manipulated, a predetermined frame rate is not exceeded.

Furthermore, the ultrasonic observation system 1 can be configured to have a function by which the videos taken only in the contrast radiography mode are considered for the TIC analysis. In that case, the mode of taking each video is stored in a corresponding manner to that video in the memory unit 40.

Moreover, the ultrasonic observation system 1 can be configured to output the result of the TIC analysis, which is performed by the TIC analysis unit 35, as CSV data.

Furthermore, the ultrasonic observation system 1 can be configured to enable the user to selectively exclude arbitrary frames from the target frames for TIC analysis.

Moreover, the ultrasonic observation system 1 can be configured to enable selection of either the frame corresponding to 0 second in the timer or the frame corresponding to the start of recording as the frame for starting the TIC analysis.

Furthermore, the ultrasonic observation system 1 can be configured to have a function by which, even if the observation target becomes misaligned, the region of interest set for the TIC analysis automatically follows the movement of the observation target.

Moreover, when a touch-pad is connected as the input device, the ultrasonic observation system 1 can be configured to enable making changes in the size of the region of interest for the TIC analysis according to pinch-in/pinch-out operations.

Furthermore, at the time of creating a graph of the result of the TIC analysis performed by the TIC analysis unit 35, the ultrasonic observation system 1 can be configured to enable adjustment of the smoothness (i.e., to enable smoothing) of the graph. The smoothing is implemented by, for example, calculating the average value of a plurality of successive frames and then drawing a graph.

Moreover, the ultrasonic observation system 1 can be configured in such a way that, at the time of performing the TIC analysis, the TIC analysis unit 35 performs thinning of the ultrasonic images at regular intervals and then performs the TIC analysis. This function enables achieving reduction in the period of time taken for the TIC analysis.

Furthermore, the ultrasonic observation system 1 can be configured to automatically calculate the rise time of the luminance in the TIC analysis (for example, the period of time taken for the luminance to reach 10% to 90% of the maximum value).

Moreover, when the display control unit 37 displays the result of the TIC analysis in the display device 4, the ultrasonic observation system 1 can be configured to display an approximated curve of Wash-in/Wash-out.

Furthermore, at the time of outputting the CSV data, the ultrasonic observation system 1 can be configured to enable storing, in tandem, the still images being displayed in the display device 4.

Moreover, the ultrasonic observation system 1 can be configured to enable storing the intermediate states during the TIC analysis performed by the TIC analysis unit 35 and enable calling the stored states.

Furthermore, the ultrasonic observation system 1 can be configured in such a way that, in the case of performing the TIC analysis in succession, when an ROI copy button is pressed (turned ON), the shape of the previously-set region of interest is copied and is used in setting the region of interest meant for performing the subsequent TIC analysis. Moreover, the ultrasonic observation system 1 can be configured to have a function by which, when the ROI copy button is again pressed (turned OFF), the shape of the region of interest is reset to the initial setting.

Moreover, the ultrasonic observation system 1 can be configured to have a function by which, when the observation target becomes misaligned, the position of the region of interest is automatically interpolated between two selected frames. For example, this interpolation is implemented by linearly shifting the position of the region of interest in small steps from the position of the region of interest in the first selected frame to the position of the region of interest in the next selected frame. Moreover, the ultrasonic observation system 1 can be configured to have a function by which, in the case of interpolating the position of the region of interest, after the second selected frame onward, the position of the region of interest is not changed and the same position is maintained.

Furthermore, the ultrasonic observation system 1 can be configured to have a function by which, in the case of interpolating the position of the region of interest, if a new frame is selected in between the selected frames, interpolation is performed between the position of the region of interest in the newly-selected frame and the positions of the regions of interest in the previous frame and the subsequent frame.

Moreover, when a touch-pad is connected as the input device, the ultrasonic observation system 1 can be configured to enable scrolling of the result of the TIC analysis by touching a trackpad using two fingers and sliding it in the vertical direction.

Furthermore, the ultrasonic observation system 1 can be configured to have a function by which, if a plurality of graphs of the TIC analysis result is displayed in the display device 4, when a position on a graph is selected, a cross-hair cursor is displayed with its vertical cursor positioned at the selected position and its horizontal cursor positioned on the closest graph to the pointer. Moreover, when a plurality of graphs is displayed in an overlapping manner, the most-recently selected graph is selected on a priority basis.

Moreover, when the images being displayed in the display device 4 are to be stored in the memory unit 40, the ultrasonic observation system 1 can be configured to enable performing setting of whether or not to store the CSV data in tandem.

Furthermore, the ultrasonic observation system 1 can be configured in such a way that, when an item on the graph of the TIC analysis result (such as the vertical axis of the graph) is double-tapped, a popup menu of the related items (such as the maximum value and the minimum value) is displayed.

Moreover, the ultrasonic observation system 1 can be configured to have a function by which, when a small-size connector is used, information enabling identification of the connected ultrasonic probe 2 (i.e., information such as the probe ID, the model name, the serial number, the vibrator type, the connector information, and the version) is obtained using the I2C communication (serial communication) via the connector to which the ultrasonic probe 2 is connected, and transmission-reception and image display is performed in the most suitable manner.

Furthermore, in the ultrasonic observation system 1, the display control unit 37 can be configured to partially synthesize pulse inversion data and B mode image data and to generate images in which the pulse inversion data is offset with the B mode image data. In that case, the position at which synthesis is to be performed can be set to be variable according to the ultrasonic probe 2 that is used.

Moreover, in the ultrasonic observation system 1, in order to transmit the information of the ultrasonic probe 2 to the ultrasonic observation device 3, a converting unit meant for converting parallel data to serial data can be installed either in the ultrasonic probe 2 or in the detachably-attachable cable used to connect the ultrasonic probe 2 to the ultrasonic observation device 3.

Furthermore, in the ultrasonic observation system 1, coded ultrasonic pulses can be transmitted to the subject being examined.

Moreover, the ultrasonic observation system 1 can be configured to have a function by which, at the time of storing images that are being displayed in the display device 4, the related measurement data is stored in the CSV format and in a corresponding manner to the image data.

Furthermore, when a touch-pad is connected as the input device, the ultrasonic observation system 1 can be configured in such a way that, if the state in which one operating finger is touching the touch-pad is continuously changed to the state in which two operating fingers are touching the touch-pad or if the state in which two operating fingers are touching the touch-pad is continuously changed to the state in which one operating finger is touching the touch-pad, the functions can be switched according to the operation.

Moreover, the ultrasonic observation system 1 can be configured to have a function enabling switching between the autofocus that is linked to the display range and the fixed focus that is set using the absolute value of the depth.

Furthermore, the ultrasonic observation system 1 can be configured to enable individually changing the focus points in the case of obtaining B mode data and in the case of obtaining THE mode data.

According to the present disclosure, it becomes possible to implement an ultrasonic observation device, and a method for operating an ultrasonic observation device that are capable of notifying about whether or not user operations affecting the analysis result were performed during the recording of the target video for analysis.

Additional advantages and modifications will readily occur to those skilled in the art. Therefore, the disclosure in its broader aspects is not limited to the specific details and the exemplary embodiments shown and described herein. Accordingly, various modifications may be made without departing from the spirit or scope of the general inventive concept as defined by the appended claims and their equivalents.

What is claimed is:

1. An ultrasonic observation device comprising:
a processor comprising hardware, the processor being configured to:
read a video made of ultrasonic images arranged in a chronological manner from a memory;
perform time intensity curve (TIC) analysis on the video based on temporal changes in signal intensities of ultrasonic waves reflected by a contrast dye administered into a subject being examined;
extract information about user operation stored in the memory along the ultrasonic images; and
control a display to display fact that the information about user operation is extracted.

2. The ultrasonic observation device according to claim 1, wherein the processor notifies operation timing at which the user operation was performed.

3. The ultrasonic observation device according to claim 2, wherein the processor superimposes the operation timing, at which the user operation was performed, onto a TIC graph area in which result of the TIC analysis is displayed as a graph, and control the display to display superimposition result.

4. The ultrasonic observation device according to claim 3, wherein the processor automatically sets, as TIC analysis range, interval between neighboring operation timings across a selected point of time in the TIC graph area.

5. The ultrasonic observation device according to claim 1, wherein the processor automatically sets, as TIC analysis range, interval from start of storing till the user operation performed for first time.

6. The ultrasonic observation device according to claim 1, wherein, according to selection made by user, the processor automatically sets, as TIC analysis range, interval from start of storing till the user operation performed for first time.

7. The ultrasonic observation device according to claim 1, wherein the display control unit notifies type of the user operation.

8. The ultrasonic observation device according to claim 7, wherein the processor notifies the ultrasonic image as well as type of the user operation.

9. The ultrasonic observation device according to claim 1, wherein the processor extracts only information that affects the ultrasonic images from the information about user operations.

10. A method for operating an ultrasonic observation device, the method comprising:
reading a video made of the ultrasonic images arranged in a chronological manner from a memory;
performing time intensity curve analysis on the video based on temporal changes in signal intensities of ultrasonic waves reflected by a contrast dye administered into a subject being examined;
extracting information about user operation stored in the memory along the ultrasonic images; and
controlling a display to display fact that the user operation is extracted.
